(19) United States
(12) Patent Application Publication
(10) Pub. No.: US 2026/0030663 A1
(43) Pub. Date: Jan. 29, 2026

(54) MARKETPLACE FOR ENABLING SUSTAINABLE CHEMISTRY PRODUCTION PROCESSES

(71) Applicant: EMD MILLIPORE CORPORATION, Burlington, MA (US)

(72) Inventors: Misael L. SILVA, Rio de Janeiro (BR); Emma GARDENER, Burlington, MA (US); Karen KARAPETYAN, Darmstadt (DE); Fernando LUENGO, St. Louis, MO (US); Claudia SAUD, Barueri (BR); Ewa GAJEWSKA, Poznan (PL)

(73) Assignee: EMD MILLIPORE CORPORATION, Burlington, MA (US)

(21) Appl. No.: 18/996,901

(22) PCT Filed: Jul. 24, 2023
(86) PCT No.: PCT/US2023/070804

§ 371 Date: Jan. 17, 2025

Related U.S. Application Data
(60) Provisional application No. 63/369,084, filed on Jul. 22, 2022.

(51) Int. Cl. G06Q 30/0601; G16C 20/10

(52) U.S. Cl. CPC G06Q 30/06212; G16C 20/10

(57) ABSTRACT

A method and a respective system for performing a chemical synthesis via a computer by Defining an organic target molecule of the chemical synthesis to be produced in the chemical production plant; Providing information describing the organic target molecule to a software-based platform being hosted by a computer connected to a network, wherein the platform holds inventories of chemical suppliers and the inventories contain information about synthetic reagents needed for the chemical synthesis; Routing the chemical synthesis via the computer, wherein the computer checks the inventories of the platform for the synthetic reagents to perform the chemical synthesis by comparing the information describing the organic target molecule and the information in the inventories and connects those chemical suppliers to the chemical production plant which provide synthetic reagents according to the compared information; and Producing the desired organic target molecule in the chemical production plant.

### BACKGROUND OF THE INVENTION

The disclosed invention relates to a method and a system for performing organic-chemical synthesis to produce an organic target molecule by using automatically determined and selected by-products and/or waste as basic starting materials.

The invention belongs to the technical fields of automated chemical synthesis, quality control, process chemistry, process optimization, cheminformatics, retrosynthesis, green chemistry and supply chain optimization.

Green or sustainable chemistry is a new approach for organic-chemical synthesis that is growing rapidly worldwide. Sustainable synthesis has been introduced as a method for environmental and economic progress and as part of the UN Sustainable Development Goals, as responsible consumption and production. In order for a synthesis to be considered green it needs to follow the principles of green chemistry, which include avoiding unneccesary waste, improving atom-economy, avoiding auxiliary substances, employing catalysts that can be used in small quantities and can be recovered and recycled, reducing energy requirements and using energy efficiently, and using renewable and bio-degradable materials. Accordingly, fine chemical suppliers are creating green alternatives to replace standard chemicals on the market. This approach is limited to a few chemicals with green alternatives, while for the majority of chemical compounds no alternative is available. The green alternatives are developed one at a time and are not always a direct replacement; they may require significant protocol adjustments for use. In general, there are two kinds of green alternatives: 1. Drop-in replacements and 2. non drop-in replacements. In the first, the replacement contains the same chemical entity, but it has been produced from renewable sources. In this case, no protocol adjustments are required. For a non drop-in replacement, the chemical compound is different and requires protocol adjustments in order to use. In recent years, an increasing number of companies have become interested in using green alternatives, but they must source each green option individually since there is no centralized market hub to consolidate the options. A network approach based on chemical supplier websites is a significant improvement because they can offer the benefits of automation, convenience, management, and enhanced consumer selection.

One problem that has arisen is that matching services are limited to searching for matches only within their own platform. Thus, only users who have gone through the website and registered products are searched for a match. One solution to this problem is to have a marketplace, where any green chemistry product available could be registered into the system. Further, the green chemistry supplier must then promote their alternatives to create traffic to the marketplace, but the lack of application protocols on intended use could generate inefficiency and may cause users to give up on the search process. An additional problem is that the search results could present many irrelevant entities to the searcher. This costs the user of the service time and may deter them from continuing to review all of the search results.

Another problem is that large numbers of unwanted communication requests can become a nuisance to the user. Users with the most attractive profiles are oftentimes the ones that receive the most unwanted attention. If the users with the most attractive profiles cease to use the system, the quality of the user pool deteriorates. The solution could be creating a platform to connect suppliers and customers, matching them based on their preferences.

To comply with and enhance such state of the art it would therefore be desirable to find an efficient method to perform a green organic-chemical synthesis which removes the shortcomings of the known state of the art.

### BRIEF SUMMARY OF THE INVENTION

This task can be solved by a method for performing a chemical synthesis via a computer comprising the following steps of Defining a desired organic target molecule of the chemical synthesis to be produced in the chemical production plant; Providing information describing the desired organic target molecule to a software-based platform being hosted by a computer connected to a network, wherein the platform holds inventories of chemical suppliers and the inventories comprises of information about synthetic reagents, like by-products and/or waste, needed for the chemical synthesis; Routing the chemical synthesis via the computer, wherein the computer checks the inventories of the platform for the necessary synthetic reagents to perform the chemical synthesis to produce the target molecule by comparing the information describing the desired organic target molecule and the information in the inventories and connects those chemical suppliers to the chemical production plant which provide the most suitable synthetic reagents according to the compared information; and producing the desired organic target molecule in the chemical production plant with the provided synthetic reagents from the chemical suppliers chosen by the computer of the software-based platform. The purpose of this approach is to provide a way to produce the target molecule of the synthesis by using by-products and/or waste etc and therefore recycle waste from other processes and/or avoid creating more waste. Via the platform many different producers can be brought together which greatly enhances the efficiency of the green synthesis approach. Another positive effect is that the software of the platform is configured in such a way that if presented with suitable information about the target molecule it can route the most efficient way to produce the molecule by using the available by-products/waste registered in the platform by the other users. In general the invention enables marketplace customers to perform a search of biproducts and/or industrial waste available from the registered/vetted suppliers/vendors in the marketplace, and further connects customers, consultants in chemical purification and chemical process optimization, and the registered suppliers/vendors seeking production cost-efficency and/or sustainability. Furthermore to ensure high quality of suppliers/vendors of byproducts a vetting process that validates customer profile, inventories, availability, amounts, country of origin, FOB/CIF price, and other is established.

Advantageous and therefore preferred further developments of this invention emerge from the associated sub claims and from the description and the associated drawings.

One of those preferred further developments of the disclosed method comprise that the defining of the target molecule, providing its information and organizing the production with the provided most suitable synthetic reagents from the chosen chemical suppliers is done either by at least one assigned user and/or by an automated system of the chemical production plant. While with the current state of the technology especially in the current production plants human users with computer support will be most common, it is also possible to configure the software-based platform in a way that it can communicate and respectively work with automatic ordering systems from the side of the customers.

Another one of those preferred further developments of the disclosed method comprise that the routing of the chemical synthesis via the computer is done by providing additional information about specific required synthetic reagents needed for the chemical synthesis which are then searched for in the platform inventory by the computer of the platform or by creating new synthetic routes to the target molecule that prioritize specified synthetic reagents as starting materials, that are available in the platform inventory. While the first option will be the standard option where the customer just provides information about the reagents needed for an already determined synthesis process, a second option comprises the possibility that the customer just describes the target molecule and lets the software of the platform find the most suitable synthesis process based on the available reagents registered at the platform, which, if possible, will then be provided to the customer together with the suggested respective synthesis process. In this case the software of the platform has to be configured respectively to provide this much more complex functionality. The latter option has the also the advantage that there is no or at least a shorter delay if not all necessary reagents for a specific synthesis process are available.

Another one of those preferred further developments of the disclosed method comprise that the software-based platform is an online marketplace platform where industry customers can register as chemical suppliers or operators of chemical production plants and upload the inventories listing their by-products and/or waste which can be used as synthetic reagents for the chemical synthesis. While the platform concept in general can be used by every customer who needs or can provide chemical basic products needed for a chemical production process, the invention primarily targets industrial customers like professional chemical suppliers and/or operators of chemical production plants who perform respective organic-chemical synthesis.

Another one of those preferred further developments of the disclosed method comprise that operators of chemical production plants can set reminders for required synthetic reagents which are not available yet by the registered chemical suppliers and the computer of the software-based platform informs the operators if the required synthetic reagents become available. This feature applies for the case that a specific synthesis process with fixed required reagents which shall at least partly be acquired via the platform has already been determined by the customer. In that case reminders which inform the customer when a necessary reagent is available provide the advantage that the customer can immediately contact the respective supplier before another customer takes it therefore reducing the waiting time and avoiding further delays for the production process of the synthesis.

Another one of those preferred further developments of the disclosed method comprise that the information describing the target molecule and the information in the inventories comprises of chemical features of the synthetic reagents like chemical structures, CAS-numbers, chemical classes, amount and volumes of the synthetic reagents available and the type of presentation which can be either characterized, purified or blended. This list is not exhaustive. It comprises only the most common information types and includes also all further suitable information types.

A further component of the claimed invention is a system for performing chemical syntheses comprising a computer connected to a network and hosting a software-based platform, a chemical production plant with at least one control computer connected to the network, wherein the system is configured to perform the following method steps of Defining a desired organic target molecule of the chemical synthesis to be produced in the chemical production plant; Providing information describing the desired organic target molecule to a software-based platform being hosted by a computer connected to a network, wherein the platform holds inventories of chemical suppliers and the inventories comprises of information about synthetic reagents, like by-products and/or waste, needed for the chemical synthesis; Routing the chemical synthesis via the computer, wherein the computer checks the inventories of the platform for the necessary synthetic reagents to perform the chemical synthesis to produce the target molecule by comparing the information describing the desired organic target molecule and the information in the inventories and connects those chemical suppliers to the chemical production plant which provide the most suitable synthetic reagents according to the compared information; and Producing the desired organic target molecule in the chemical production plant with the provided synthetic reagents from the chemical suppliers chosen by the computer of the software-based platform.

Another component of the disclosed invention is a Computer program comprising instructions which cause the involved computers to carry out the following method steps of Defining a desired organic target molecule of the chemical synthesis to be produced in the chemical production plant; Providing information describing the desired organic target molecule to a software-based platform being hosted by a computer connected to a network, wherein the platform holds inventories of chemical suppliers and the inventories comprises of information about synthetic reagents, like by-products and/or waste, needed for the chemical synthesis; Routing the chemical synthesis via the computer, wherein the computer checks the inventories of the platform for the necessary synthetic reagents to perform the chemical synthesis to produce the target molecule by comparing the information describing the desired organic target molecule and the information in the inventories and connects those chemical suppliers to the chemical production plant which provide the most suitable synthetic reagents according to the compared information; and Producing the desired organic target molecule in the chemical production plant with the provided synthetic reagents from the chemical suppliers chosen by the computer of the software-based platform. The program parts responsible for the single method steps are running on the respective computer parts. How the program itself is partitioned depends on the computer hardware being involved. It is possible to use a main software running on one of the mentioned computers or a separate computer which controls local client programs. Other options include equal instances of the software who communicate with each other and so on.

Only requirement for this computer program to perform the whole method as described is, that the used program and its respective hardware components are able to perform the method completely and automatically. Such a program can then be stored on a Computer-readable storage medium and/or data carrier signal which cause the involved computers to carry out the previously disclosed method steps. The storage medium can be stored on any suitable digital memory like an usb drive, a harddisk, a flashdrive and so on. From that memory it can also be provided via remote communication means using respective data carrier signals, like ethernet, wired or wireless, or any other suitable network transmission means, for transmitting the software to its target hardware.

### BRIEF DESCRIPTION OF SEVERAL VIEWS OF THE DRAWINGS

FIG. 1: a schematic overview about the involved system components

FIG. 2: a general overview about the necessary method steps

FIG. 3: an exemplary organic target molecule of a chemical synthesis

### DETAILED DESCRIPTION OF THE INVENTION

The invention will be explained in more detail by presenting a preferred exemplary embodiment which disclose preferred ways to perform the invented method for performing a green chemical synthesis.

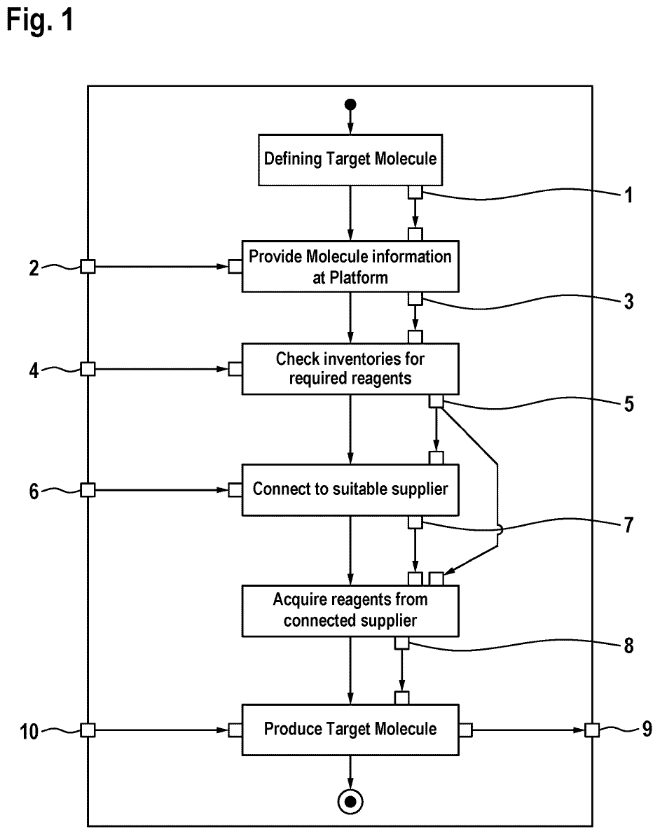

The invented system which performs the invented method, including the software-based marketplace 3 is shown in FIG. 1. It comprises of an inventory server, usually realized via a kind of computer which is suitable for being used as a server, which hosts an application programming interface (API). This API enables users 6, 10 for the marketplace 3, on the one hand chemical by-product suppliers 6 and on the other hand chemical manufacturers 10, to get a specific access to the marketplace realized via the inventory server. For the suppliers 6 a specific Inventory Deposition Interface (IDI) provides access, e.g. in form a suitable Graphical User Interface (GUI). Another option is that the IDI provides just a mere data exchange interface so the suppliers 6 can connect to the IDI with their own ERP tools on their own computers. It is also possible that both GUI and ERP interface are provided. The manufacturer clients can access the marketplace with their respective computers via a Marketplace Browsing Interface (MBI), which preferably uses a GUI. Those clients can be further supported by specific consultants who are Designers of Separation and Purification Services. As previously explained the system components can also differ from embodiment to embodiment. Especially the kind of involved computers can differ greatly, depending on how much of the steps is performed by human users with the help of computers and application software or done automatically by specific computers using for instance Al based software algorithms.

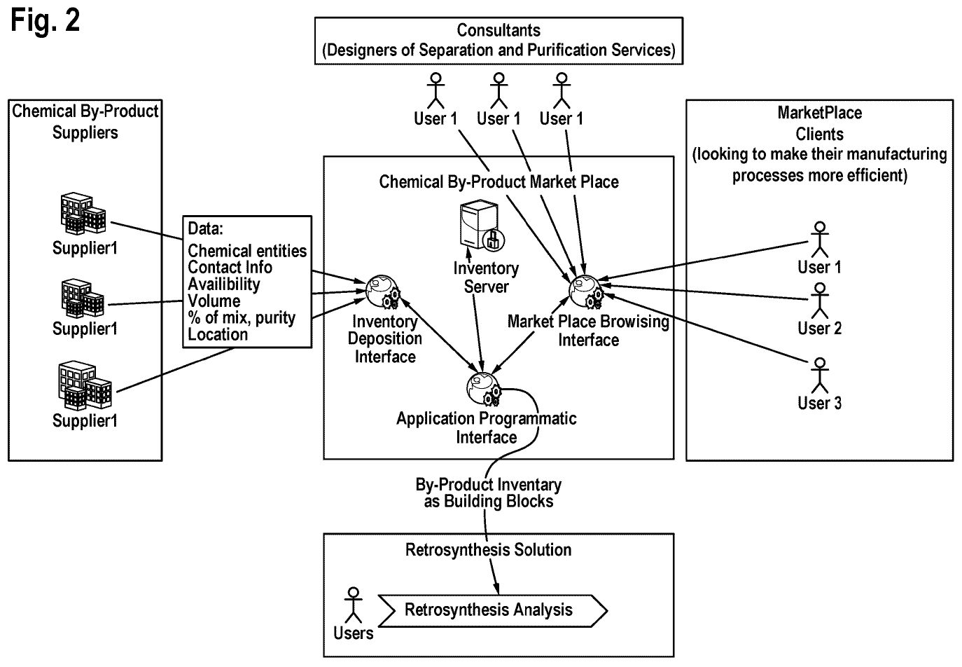

FIG. 2 shows now a generalized overview about the necessary method steps in all possible preferred embodiment. The steps itself are performed divergent in every exemplary embodiment dependent on the different conditions. First a target molecule 1 which a user (manufacturer) 10 wants to produce with the respective chemical synthesis needs to be defined by this manufacturer 10. To do so the manufacturer 10 also needs to provide specific molecule information 2. With this information 2 the Marketplace 3 checks the inventories 4 of all registered suppliers 6 if they have reagents 5 available which are required for the specific synthesis which the manufacturer 10 wants to perform to produce the target molecule 1. That step can also include in another specific embodiment that the marketplace 3 automatically and/or supported by the specific consultants determines an alternative synthesis process which also produces the target molecule 1, but with different, available reagents 5. This is especially preferred if the required reagents 5 for the original synthesis are not available in the registered suppliers inventory 4. When the required reagents 5 have been found in the inventory 4, the marketplace 3 establishes a connection 7 between the user 10 and a suitable supplier 6. The manufacturer 10 can then acquire the respective reagents 5 from the supplier 6 and use the provided reagents 8 in the synthesis process to produce the target molecule 1, 9.

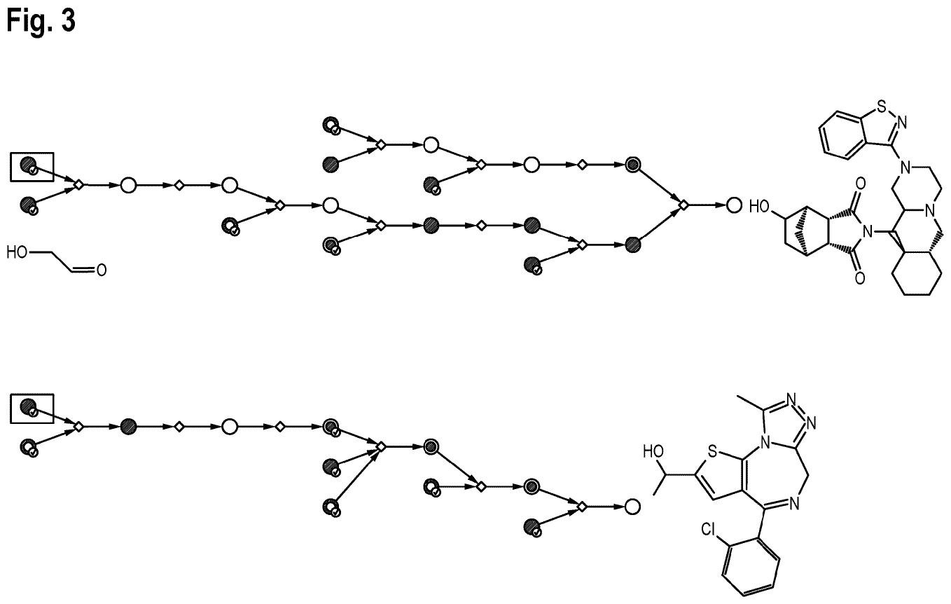

FIG. 3 discloses now a specific example of the described preferred method about how the Marketplace can be used to find uses for chemical waste by-products as starting materials, meaning the required reagents 5, for high-value chemicals, the target molecules 1, via new synthetic routes. In this example, hydroxyacetaldehyde (CAS: 141-46-8) is a waste component that was added to the inventory database 4 as a possible starting material 5. Retrosynthesis search for two different Cerilliant products, a) alpha-hydroxyetizolam (CAS: 64546-10-7) and b) 5-beta-Hydroxy Lurasidone produced routes starting from the inventory compound glycolaldehyde.

### LIST OF REFERENCES

1 Target Molecule
2 Molecule Information
3 Digital platform
4 Supplier Inventories
5 Reagents
6 Supplier
7 Supplier-Reagent-Connection
8 Provided reagents
9 Produced Target Molecule
10 Manufacturer

1. A method for performing a chemical synthesis via a computer comprising the following steps:
Defining a desired organic target molecule of the chemical synthesis to be produced in the chemical production plant;
Providing information describing the desired organic target molecule to a software-based platform being hosted by a computer connected to a network, wherein the platform holds inventories of chemical suppliers and the inventories comprises of information about synthetic reagents, like by-products and/or waste, needed for the chemical synthesis;
Routing the chemical synthesis via the computer, wherein the computer checks the inventories of the platform for the necessary synthetic reagents to perform the chemical synthesis to produce the target molecule by comparing the information describing the desired organic target molecule and the information in the inventories and connects those chemical suppliers to the chemical production plant which provide the most suitable synthetic reagents according to the compared information;
Producing the desired organic target molecule in the chemical production plant with the provided synthetic reagents from the chemical suppliers chosen by the computer of the software-based platform.

2. The method of claim 1, wherein the defining of the target molecule, providing its information and organizing the production with the provided most suitable synthetic reagents from the chosen chemical suppliers is done either by at least one assigned user and/or by an automated system of the chemical production plant.

3. The method of claim 1, wherein the routing the chemical synthesis via the computer is done by providing additional information about specific required synthetic reagents needed for the chemical synthesis which are then searched for in the platform inventory by the computer of the platform or by creating new synthetic routes to the target molecule that prioritize specified synthetic reagents as starting materials, that are available in the platform inventory.

4. The method of claim 1, wherein as the software-based platform an online marketplace platform is used where industry customers can register as chemical suppliers or operators of chemical production plants and upload the inventories listing their by-products and/or waste which can be used as synthetic reagents for the chemical synthesis.

5. The method of claim 4, wherein operators of chemical production plants can set reminders for required synthetic reagents which are not available yet by the registered chemical suppliers and the computer of the software-based platform informs the operators if the required synthetic reagents become available.

6. The method of claim 1, wherein the information describing the target molecule and the information in the inventories comprises of chemical features of the synthetic reagents like chemical structures, CAS-numbers, chemical classes, amount and volumes of the synthetic reagents available and the type of presentation which can be either characterized, purified or blended.

7. A System (12) for performing chemical synthesis comprising a service computer connected to a network and hosting a software-based platform, a chemical production plant with at least one control computer connected to the network, wherein the system is configured to perform the following method steps:
Defining a desired organic target molecule of the chemical synthesis to be produced in the chemical production plant;
Providing information describing the desired organic target molecule to the software-based platform, wherein the platform holds inventories of chemical suppliers and the inventories comprises of information about synthetic reagents, like by-products and/or waste, needed for the chemical synthesis;
Routing the chemical synthesis via the service computer, wherein the service computer checks the inventories of the platform for the necessary synthetic reagents to perform the chemical synthesis to produce the target molecule by comparing the information describing the desired organic target molecule and the information in the inventories and connects those chemical suppliers to the chemical production plant which provide the most suitable synthetic reagents according to the compared information;
Producing the desired organic target molecule in the chemical production plant via the at least one control computer by using the provided synthetic reagents from the chemical suppliers connected by the service computer of the software-based platform.

8. A Computer program comprising instructions which cause the involved computers to carry out the following method steps:
Defining a desired organic target molecule of the chemical synthesis to be produced in the chemical production plant;
Providing information describing the desired organic target molecule to a software-based platform being hosted by a computer connected to a network, wherein the platform holds inventories of chemical suppliers and the inventories comprises of information about synthetic reagents, like by-products and/or waste, needed for the chemical synthesis;
Routing the chemical synthesis via the computer, wherein the computer checks the inventories of the platform for the necessary synthetic reagents to perform the chemical synthesis to produce the target molecule by comparing the information describing the desired organic target molecule and the information in the inventories and connects those chemical suppliers to the chemical production plant which provide the most suitable synthetic reagents according to the compared information;
Producing the desired organic target molecule in the chemical production plant with the provided synthetic reagents from the chemical suppliers chosen by the computer of the software-based platform.

9. A Computer-readable storage medium and/or data carrier signal having stored thereon the computer program of claim 8 which cause the involved computers to carry out the following method steps:
Defining a desired organic target molecule of the chemical synthesis to be produced in the chemical production plant;
Providing information describing the desired organic target molecule to a software-based platform being hosted by a computer connected to a network, wherein the platform holds inventories of chemical suppliers and the inventories comprises of information about synthetic reagents, like by-products and/or waste, needed for the chemical synthesis;
Routing the chemical synthesis via the computer, wherein the computer checks the inventories of the platform for the necessary synthetic reagents to perform the chemical synthesis to produce the target molecule by comparing the information describing the desired organic target molecule and the information in the inventories and connects those chemical suppliers to the chemical production plant which provide the most suitable synthetic reagents according to the compared information;
Producing the desired organic target molecule in the chemical production plant with the provided synthetic reagents from the chemical suppliers chosen by the computer of the software-based platform.
